(19) United States
(12) Patent Application Publication
(10) Pub. No.: US 2010/0121004 A1
(43) Pub. Date: May 13, 2010

(54) PURIFICATION PROCESS FOR SEMICONDUCTING MONOMERS

(75) Inventors: Yuning Li, Singapore (SG); Ping Liu, Mississauga (CA); Yiliang Wu, Mississauga (CA)

Correspondence Address: FAY SHARPE / XEROX - ROCHESTER, 1228 EUCLID AVENUE, 5TH FLOOR, THE HALLE BUILDING, CLEVELAND, OH, 44115, US

(73) Assignee: XEROX CORPORATION, Norwalk, CT (US)

(21) Appl. No.: 12/270,088

(22) Filed: Nov. 13, 2008

(51) Int. Cl. C08F 4/26; C07D 333/50; C08F 28/06

(52) U.S. Cl. 526/90; 549/43; 526/256

(57) ABSTRACT

Disclosed is a process for purifying monomers of Formula (II):

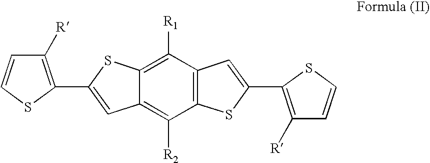

wherein R₁ and R₂ are independently selected from alkyl, substituted alkyl, aryl, substituted aryl, alkoxy, substituted alkoxy, and halogen; and R′ is selected from hydrogen, alkyl, substituted alkyl, aryl, substituted aryl, alkoxy, substituted alkoxy, and halogen. After the monomer is synthesized, it is purified by column chromatography using neutral alumina and hexane as an eluent. The resulting product can also be further recrystallized using isopropanol, hexane, heptane, or toluene. Polymers formed from the purified monomer exhibit higher mobility and increased reproducibility of the mobility.

### BACKGROUND

The present disclosure relates, in various embodiments, to processes for purifying compositions used in electronic devices, such as thin film transistors (“TFT”s). The present disclosure also relates to the components or layers produced using such compositions and processes, as well as electronic devices containing such materials.

Thin film transistors (TFTs) are fundamental components in modern-age electronics, including, for example, sensors, image scanners, and electronic display devices. It is generally desired to make TFTs which have not only much lower manufacturing costs, but also appealing mechanical properties such as being physically compact, lightweight, and flexible.

TFTs are generally composed of a supporting substrate, three electrically conductive electrodes (gate, source and drain electrodes), a channel semiconducting layer, and an electrically insulating gate dielectric layer separating the gate electrode from the semiconducting layer. In organic TFTs, one or more of these compounds is formed from an organic compound, such as an organic polymer.

It is desirable to improve the performance of known TFTs. Performance can be measured by at least two properties: the mobility and the on/off ratio. The mobility is measured in units of cm²/V·sec; higher mobility is desired. The on/off ratio is the ratio between the amount of current that leaks through the TFT in the off state versus the current that runs through the TFT in the on state. Typically, a higher on/off ratio is more desirable.

Some semiconducting polymers suitable for use in an organic TFT have the structure of Formula (I):

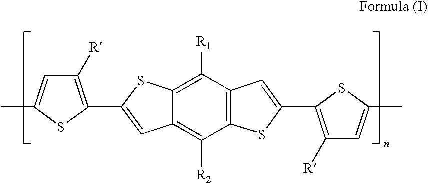

wherein R₁ and R₂ are independently selected from alkyl, substituted alkyl, aryl, substituted aryl, alkoxy, substituted alkoxy, and halogen; and R′ is selected from hydrogen, alkyl, substituted alkyl, aryl, substituted aryl, alkoxy, substituted alkoxy, and halogen. These semiconducting polymers are disclosed in U.S. patent application Ser. No. 11/586,449, filed on Oct. 25, 2006. That application is hereby fully incorporated by reference herein.

However, the semiconducting polymers typically had mobilities below 0.2 cm²/V·sec. In addition, the mobilities were difficult to reproduce. This difficulty in reproducibility and low mobility may be attributed to impurities in the polymers, which arise from impurities in the monomer used to form the polymers.

It is desirable to provide processes that further purify monomers used to form semiconducting polymers. Improved purity would provide better reproducibility of mobility results as well as higher mobilities.

### BRIEF DESCRIPTION

Disclosed in embodiments are processes for purifying monomers, such as those typically used in semiconducting polymers.

Disclosed in some embodiments is a process for purifying a monomer of Formula (II):

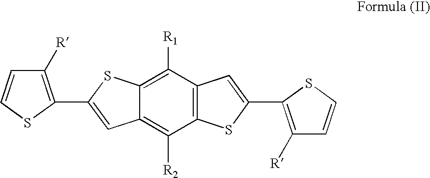

wherein R₁ and R₂ are independently selected from alkyl, substituted alkyl, aryl, substituted aryl, alkoxy, substituted alkoxy, and halogen; and R′ is selected from hydrogen, alkyl, substituted alkyl, aryl, substituted aryl, alkoxy, substituted alkoxy, and halogen; the process comprising: providing a starting mixture containing the monomer of Formula (II) and impurities; and passing the starting mixture through a column containing alumina using a non-polar solvent as an eluent to separate the impurities from the monomer of Formula (II).

The process may further comprise recrystallizing the monomer of Formula (II) from isopropanol, hexane, heptane, or toluene.

The non-polar solvent may be hexane. The alumina may be neutral alumina. In some embodiments, the column consists essentially of neutral alumina.

R₁ and R₂ may be identical to each other. Alternatively R₁, R₂, and R′ may be identical to each other and are straight chain alkyl having from about 8 to about 18 carbon atoms. In some instances, R₁, R₂, and R′ are C₁₂H₂₅.

The resulting monomer of Formula (II) may have a purity of 98% or greater, including a purity of 99.5% or greater.

In other embodiments is disclosed a process for preparing a semiconducting polymer with improved mobility, comprising: providing a starting mixture comprising impurities and a monomer of Formula (II):

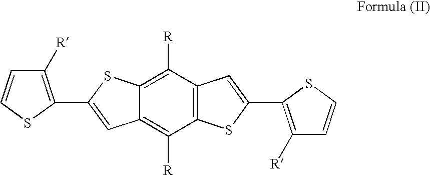

wherein R₁ and R₂ are independently selected from alkyl, substituted alkyl, aryl, substituted aryl, alkoxy, substituted alkoxy, and halogen; and R′ is selected from hydrogen, alkyl, substituted alkyl, aryl, substituted aryl, alkoxy, substituted alkoxy, and halogen; passing the starting mixture through a column containing alumina using hexane as an eluent to separate the impurities from the monomer of Formula (II); and polymerizing the monomer of Formula (II) to obtain the semiconducting polymer of Formula (I):

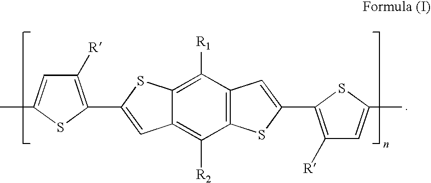

In still other embodiments is disclosed a process for purifying a BTBT-12 monomer:

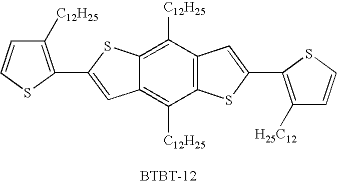

the process comprising: providing a starting mixture containing the BTBT-12 monomer and impurities; passing the starting mixture through a column containing neutral alumina using only a non-polar solvent such as hexane as an eluent to separate the impurities from the BTBT-12 monomer; and recrystallizing the BTBT-12 monomer using a solvent such as isopropanol, hexane, heptane, or toluene.

Also included in further embodiments are the semiconducting polymers, semiconducting layers, and/or thin film transistors incorporating the monomers and polymers produced by the disclosed processes.

These and other non-limiting characteristics of the exemplary embodiments of the present disclosure are more particularly described below.

### BRIEF DESCRIPTION OF THE DRAWINGS

The following is a brief description of the drawings, which are presented for the purpose of illustrating the exemplary embodiments disclosed herein and not for the purpose of limiting the same.

FIG. 1 is a first exemplary embodiment of an OTFT of the present disclosure.

FIG. 2 is a second exemplary embodiment of an OTFT of the present disclosure.

FIG. 3 is a third exemplary embodiment of an OTFT of the present disclosure.

FIG. 4 is a fourth exemplary embodiment of an OTFT of the present disclosure.

FIG. 5 is the MALDI-TOF spectrum for the product of Comparative Example 1 (collected after being run through the column).

FIGS. 6A-6D are illustrations of the impurities found in Comparative Example 1.

FIG. 7 is the MALDI-TOF spectrum for the product of Example 1 (collected after being run through the column).

FIG. 8 is the MALDI-TOF spectrum for the impurities recovered from the column of Example 1.

### DETAILED DESCRIPTION

A more complete understanding of the components, processes, and apparatuses disclosed herein can be obtained by reference to the accompanying figures. These figures are merely schematic representations based on convenience and the ease of demonstrating the present development and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

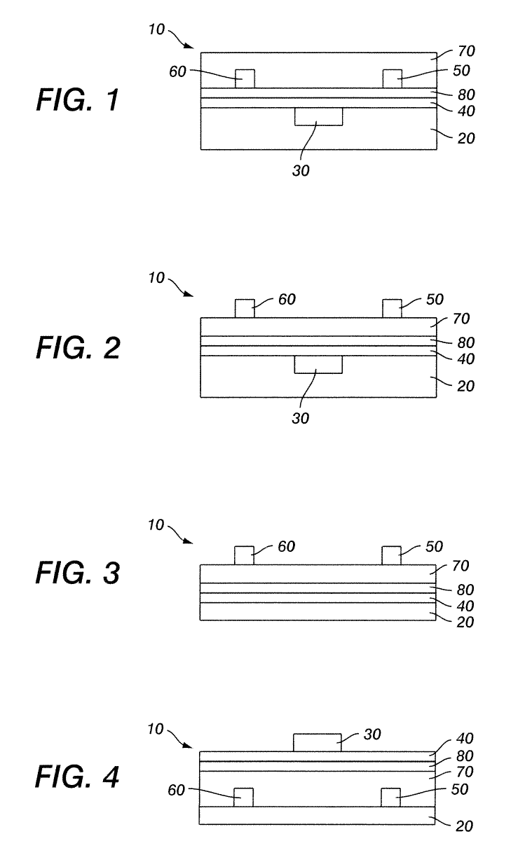

FIG. 1 illustrates a first OTFT embodiment or configuration. The OTFT 10 comprises a substrate 20 in contact with the gate electrode 30 and a dielectric layer 40. Although here the gate electrode 30 is depicted within the substrate 20, this is not required. However, of some importance is that the dielectric layer 40 separates the gate electrode 30 from the source electrode 50, drain electrode 60, and the semiconducting layer 70. The source electrode 50 contacts the semiconducting layer 70. The drain electrode 60 also contacts the semiconducting layer 70. The semiconducting layer 70 runs over and between the source and drain electrodes 50 and 60. Interfacial layer 80 is located between dielectric layer 40 and semiconducting layer 70.

FIG. 2 illustrates a second OTFT embodiment or configuration. The OTFT 10 comprises a substrate 20 in contact with the gate electrode 30 and a dielectric layer 40. The semiconducting layer 70 is placed over or on top of the dielectric layer 40 and separates it from the source and drain electrodes 50 and 60. Interfacial layer 80 is located between dielectric layer 40 and semiconducting layer 70.

FIG. 3 illustrates a third OTFT embodiment or configuration. The OTFT 10 comprises a substrate 20 which also acts as the gate electrode and is in contact with a dielectric layer 40. The semiconducting layer 70 is placed over or on top of the dielectric layer 40 and separates it from the source and drain electrodes 50 and 60. Interfacial layer 80 is located between dielectric layer 40 and semiconducting layer 70.

FIG. 4 illustrates a fourth OTFT embodiment or configuration. The OTFT 10 comprises a substrate 20 in contact with the source electrode 50, drain electrode 60, and the semiconducting layer 70. The semiconducting layer 70 runs over and between the source and drain electrodes 50 and 60. The dielectric layer 40 is on top of the semiconducting layer 70. The gate electrode 30 is on top of the dielectric layer 40 and does not contact the semiconducting layer 70. Interfacial layer 80 is located between dielectric layer 40 and semiconducting layer 70.

The semiconducting layer may comprise the semiconducting polymer of Formula (I):

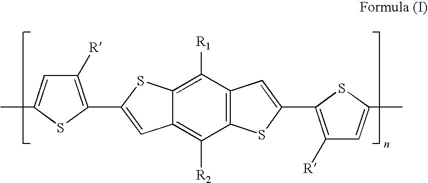

wherein R₁ and R₂ are independently selected from alkyl, substituted alkyl, aryl, substituted aryl, alkoxy, substituted alkoxy, and halogen; R′ is selected from hydrogen, alkyl, substituted alkyl, aryl, substituted aryl, alkoxy, substituted alkoxy, and halogen; and n is the degree of polymerization and can vary from 2 to about 2,500. The semiconducting polymer may have a weight average molecular weight of from about 800 to about 500,000, including from about 1,500 to about 200,000, as measured by gel permeation chromatography using polystyrene standards. This semiconducting polymer is also known as a poly(benzo[1,2-b:4,5-b′]dithiophene-co-bithiophene), or a benzodithiophene-thiophene copolymer.

Desirably, R₁, R₂, and R′ are independently alkyl. The alkyl, aryl, and alkoxy groups may be substituted with, for example, alkyl, hydroxyl, and halogen groups. In some embodiments, R₁ and R₂ are identical to each other. In others, R₁, R₂, and R′ are identical to each other and are straight chain alkyl having from about 8 to about 18 carbon atoms.

In some particular embodiments, the semiconducting polymer is that of PBTBT-12:

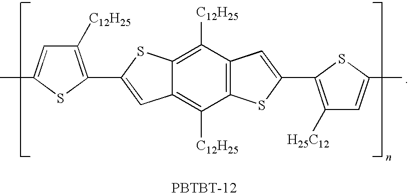

The polymers of Formula (I) are synthesized from monomers of Formula (II):

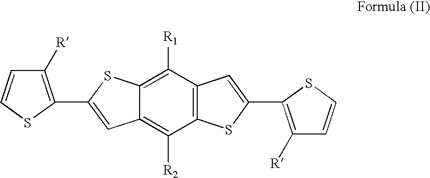

wherein R₁ and R₂ are independently selected from alkyl, substituted alkyl, aryl, substituted aryl, alkoxy, substituted alkoxy, and halogen; and R′ is selected from hydrogen, alkyl, substituted alkyl, aryl, substituted aryl, alkoxy, substituted alkoxy, and halogen. Desirably, R₁, R₂, and R′ are independently alkyl. The alkyl, aryl, and alkoxy groups may be substituted with, for example, alkyl, hydroxyl, and halogen groups.

In some embodiments, R₁ and R₂ are identical to each other. In others, R₁, R₂, and R′ are identical to each other and are straight chain alkyl having from about 8 to about 18 carbon atoms.

In some particular embodiments, the monomer is BTBT-12:

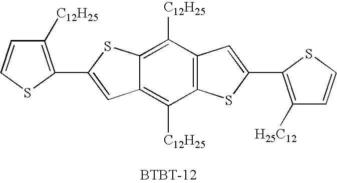

BTBT-12 is also known as 4,8-didodecyl-2,6-bis(3-dodecylthiophen-2-yl)benzo[1,2-b:4,5-b′]dithiophene.

Previous processes for purifying the monomer of Formula (II) used column chromatography, where the column contained silica gel. However, these processes produced monomer having purities below 98%. As the impurities were incorporated into the polymer chains, poor mobility and poor reproducibility resulted. The processes of the present disclosure result in monomer having purities greater than 98%, and in some embodiments greater than 99.5%.

The processes of the present disclosure comprise (a) providing a starting mixture containing the monomer of Formula (II) and impurities; and (b) passing the starting mixture through a column containing alumina using a non-polar solvent, such as hexane, as an eluent to separate the impurities from the monomer of Formula (II). The monomer can additionally be recrystallized using a solvent such as isopropanol, hexane, heptane, or toluene.

The column typically contains alumina for interaction with the impurities, though other incidental materials may also be present in the column. Desirably, neutral alumina is used. In particular embodiments, the eluent is a single compound; in other words, the eluent is not a mixture of multiple solvents. In specific embodiments, the eluent used is hexane.

Polymers made using the monomers of Formula (II) that are purified using the processes of the present disclosure have higher mobility. In embodiments, they have a mobility of 0.2 cm²/V·or greater. The polymer may also have a melting point of about 286° C. or greater, compared to a melting point of 279° C. using previous processes. This higher melting point indicates more ordered molecular packing, a crucial property for charge transport.

The semiconducting polymers can be formed from monomers of Formula (II) as seen in Scheme 1 below:

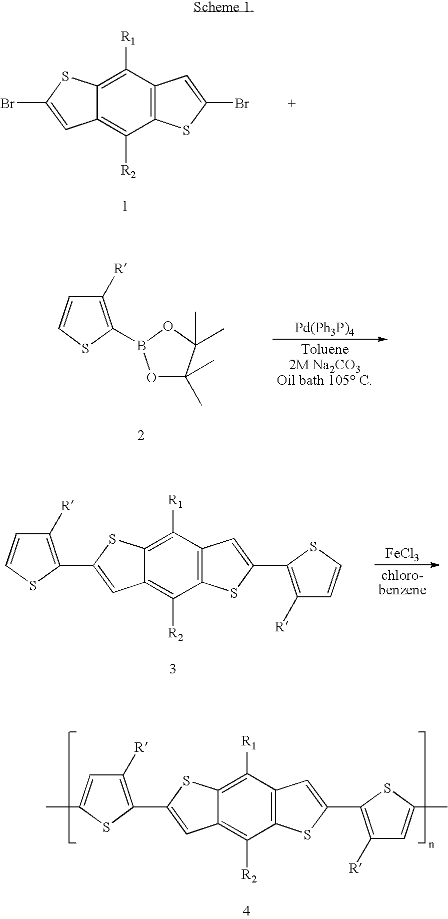

Generally, a 2,6-dibromo-4,8-disubstitutedbenzo[1,2-b:4,5-b′]dithiophene 1 is reacted with a 3-substitutedthiophene-2-boronic acid pinacol ester 2 to obtain the monomer 3. The monomer 3 is then polymerized to form the semiconducting polymer 4.

The substrate may be composed of materials including but not limited to silicon, glass plate, plastic film or sheet. For structurally flexible devices, plastic substrate, such as for example polyester, polycarbonate, polyimide sheets and the like may be used. The thickness of the substrate may be from about 10 micrometers to over 10 millimeters with an exemplary thickness being from about 50 micrometers to about 5 millimeters, especially for a flexible plastic substrate and from about 0.5 to about 10 millimeters for a rigid substrate such as glass or silicon.

The gate electrode is composed of an electrically conductive material. It can be a thin metal film, a conducting polymer film, a conducting film made from conducting ink or paste or the substrate itself, for example heavily doped silicon. Examples of gate electrode materials include but are not restricted to aluminum, gold, silver, chromium, indium tin oxide, conductive polymers such as polystyrene sulfonate-doped poly(3,4-ethylenedioxythiophene) (PSS-PEDOT), and conducting ink/paste comprised of carbon black/graphite or silver colloids. The gate electrode can be prepared by vacuum evaporation, sputtering of metals or conductive metal oxides, conventional lithography and etching, chemical vapor deposition, spin coating, casting or printing, or other deposition processes. The thickness of the gate electrode ranges from about 10 to about 500 nanometers for metal films and from about 0.5 to about 10 micrometers for conductive polymers.

The dielectric layer generally can be an inorganic material film, an organic polymer film, or an organic-inorganic composite film. Examples of inorganic materials suitable as the dielectric layer include silicon oxide, silicon nitride, aluminum oxide, barium titanate, barium zirconium titanate and the like. Examples of suitable organic polymers include polyesters, polycarbonates, poly(vinyl phenol), polyimides, polystyrene, polymethacrylates, polyacrylates, epoxy resin and the like. The thickness of the dielectric layer depends on the dielectric constant of the material used and can be, for example, from about 10 nanometers to about 500 nanometers. The dielectric layer may have a conductivity that is, for example, less than about 10⁻¹² Siemens per centimeter (S/cm). The dielectric layer is formed using conventional processes known in the art, including those processes described in forming the gate electrode.

Typical materials suitable for use as source and drain electrodes include those of the gate electrode materials such as gold, silver, nickel, aluminum, platinum, conducting polymers, and conducting inks. In specific embodiments, the electrode materials provide low contact resistance to the semiconductor. Typical thicknesses are about, for example, from about 40 nanometers to about 1 micrometer with a more specific thickness being about 100 to about 400 nanometers. The OTFT devices of the present disclosure contain a semiconductor channel. The semiconductor channel width may be, for example, from about 5 micrometers to about 5 millimeters with a specific channel width being about 100 micrometers to about 1 millimeter. The semiconductor channel length may be, for example, from about 1 micrometer to about 1 millimeter with a more specific channel length being from about 5 micrometers to about 100 micrometers.

The source electrode is grounded and a bias voltage of, for example, about 0 volt to about 80 volts is applied to the drain electrode to collect the charge carriers transported across the semiconductor channel when a voltage of, for example, about +10 volts to about −80 volts is applied to the gate electrode. The electrodes may be formed or deposited using conventional processes known in the art.

If desired, the semiconducting layer may further comprise another organic semiconductor material. Examples of other organic semiconductor materials include but are not limited to acenes, such as anthracene, tetracene, pentacene, and their substituted derivatives, perylenes, fullerenes, oligothiophenes, other semiconducting polymers such as triarylamine polymers, polyindolocarbazole, polycarbazole, polyacenes, polyfluorene, polythiophenes and their substituted derivatives, phthalocyanines such as copper phthalocyanines or zinc phthalocyanines and their substituted derivatives.

The semiconducting layer is from about 5 nm to about 1000 nm thick, especially from about 10 nm to about 100 nm thick. The semiconducting layer can be formed by any suitable method. However, the semiconducting layer is generally formed from a liquid composition, such as a dispersion or solution, and then deposited onto the substrate of the transistor. Exemplary deposition methods include liquid deposition such as spin coating, dip coating, blade coating, rod coating, screen printing, stamping, ink jet printing, and the like, and other conventional processes known in the art.

If desired, a barrier layer may also be deposited on top of the TFT to protect it from environmental conditions, such as light, oxygen and moisture, etc. which can degrade its electrical properties. Such barrier layers are known in the art and may simply consist of polymers.

The various components of the OTFT may be deposited upon the substrate in any order, as is seen in the Figures. The term “upon the substrate” should not be construed as requiring that each component directly contact the substrate. The term should be construed as describing the location of a component relative to the substrate. Generally, however, the gate electrode and the semiconducting layer should both be in contact with the dielectric layer. In addition, the source and drain electrodes should both be in contact with the semiconducting layer. The semiconducting polymer formed by the methods of the present disclosure may be deposited onto any appropriate component of an organic thin-film transistor to form a semiconducting layer of that transistor.

The following examples illustrate the devices, polymers, monomers, and methods of the present disclosure. The examples are merely illustrative and are not intended to limit the present disclosure with regard to the materials, conditions, or process parameters set forth therein.

### EXAMPLES

### COMPARATIVE EXAMPLE

A PBTBT-12 polymer was made according to conventional processes. The description below refers to Scheme 1.

Synthesis of Monomer 3:

2,6-dibromo-4,8-didodecylbenzo[1,2-b;4,5;b′]dithiophene 1 was prepared as described in Chem. Mater., 2006, Vol. 18, No. 14, pp. 3237-41, the disclosure of which is totally incorporated herein by reference.

3-dodecylthiophene-2-boronic acid pinacol ester 2 was prepared as described in U.S. Patent Publication No. 2008/0103286, the disclosure of which is totally incorporated herein by reference.

2.0 grams of dithiophene 1, 2.76 grams of pinacol ester 2, and 25 mL of toluene were added to a 250 mL 3-necked reaction flask. The resulting mixture was thoroughly stirred and was purged with argon. Next, 0.07 grams of tetrakis(triphenylphosphine palladium(0)) (Pd(Ph₃P)₄), 0.72 grams of ALIQUAT® in 10 mL toluene, and 8.4 mL of 2 M aqueous Na₂CO₃ was added to the mixture. The reaction mixture obtained was stirred at 105° C. for 72 hours. After cooling to room temperature (about 23 to about 26° C.), 200 mL of toluene was added. The resulting organic layer was collected and washed with deionized water 3 times in a separatory funnel, dried over anhydrous MgSO₄, and filtered.

After removing the solvent, the remaining solid was purified by column chromatography on silica gel (eluent: hexane/toluene, 7/1, v/v) and recrystallized from 2-propanol to yield yellow needle-like crystals. Yield: 2.4 grams (80%)

¹H NMR (CDCl₃, 300 MHz, ppm): δ 7.44 (s, 2H), 7.28 (d, J=5.1 Hz, 2H), .7.01 (d, J=5.1 Hz, 2H), 3.16 (t, 4H), 2.90 (t, 4H), 2.90 (t, 4H), 1.88 (m, 4H), 1.72 (m, 4H), 1.26 (br, 72H), 0.89 (t, 6H).

¹³C NMR (CDCl₃, 300 MHz, ppm): δ 141.15, 137.99, 136.72, 136.15, 131.58, 128.84, 125.04, 120.56, 33.82, 32.34, 31.27, 30.44, 30.12, 30.08, 30.05, 29.99, 29.94, 29.87, 29.78, 23.10, 14.52.

Synthesis of Polymer 4:

A solution of the 4,8-didodecyl-2,6-bis-(3-dodecyl-thiophen-2-yl)-benzo[1,2-b;4,5-b′]dithiophene monomer 3 (0.40 grams) in 10 mL of chlorobenzene was prepared. 0.32 grams of FeCl₃ and 10 mL of chlorobenzene were placed in a 50 mL round-bottom flask under an argon atmosphere and stirred. While stirring, the solution was added drop-wise through a dropping funnel to the well-stirred mixture over a period of 1 minute. The resulting mixture was stirred at room temperature (about 23 to about 26° C.) for 4 hours under an argon blanket. 15 mL chlorobenzene was added and the solution was put into 200 mL methanol to remove the FeCl₃. The mixture was ultrasonicated for 2 minutes, then stirred at room temperature for 1 hour. The polymer was filtered out of the mixture.

The polymer was then added to a well stirred aqueous solution of 200 mL methanol and 50 mL ammonia (30%). The mixture was subjected to ultrasonication for 30 minutes and then stirred at room temperature for 18 hours. A dark red precipitate was obtained after filtration, which was purified by Soxhlet extraction with methanol for 4 hours and heptane for 24 hours. Chlorobenzene was then used to extract the polymer for 4 hours and a red solution was obtained. Removal of the solvent resulted in 0.32 grams of poly(4,8-didodecyl-2,6-bis-(3-dodecyl-thiophen-2-yl)-benzo[1,2-b;4,5-b′]dithiophene) 4 as a dark red solid. Yield: 80%. The melting point, as measured by DSC, was 279° C.

### Example 1

A PBTBT-12 polymer was made according to the processes of the present disclosure.

Synthesis of Monomer 3:

2,6-dibromo-4,8-didodecylbenzo[1,2-b;4,5;b′]dithiophene 1 was prepared as described in Chem. Mater., 2006, Vol. 18, No. 14, pp. 3237-41, the disclosure of which is totally incorporated herein by reference.

3-dodecylthiophene-2-boronic acid pinacol ester 2 was prepared as described in U.S. Patent Publication No. 2008/0103286, the disclosure of which is totally incorporated herein by reference.

2.0 grams of dithiophene 1, 2.76 grams of pinacol ester 2, and 25 mL of toluene were added to a 250 mL 3-necked reaction flask. The resulting mixture was thoroughly stirred and was purged with argon. Next, 0.07 grams of tetrakis(triphenylphosphine palladium(0)) (Pd(Ph₃P)₄), 0.72 grams of ALIQUAT® in 10 mL toluene, and 8.4 mL of 2 M aqueous Na₂CO₃ was added to the mixture. The reaction mixture obtained was stirred at 105° C. for 72 hours. After cooling to room temperature (about 23 to about 26° C.), 200 mL of toluene was added. The resulting organic layer was collected and washed with deionized water 3 times in a separatory funnel, dried over anhydrous MgSO₄, and filtered.

After removing the solvent, the remaining solid was purified by column chromatography on neutral alumina (obtained from Sigma-Aldrich) (eluent: hexane) and recrystallized from isopropanol to yield light yellow crystals. Yield: 2.1 g (70%).

¹H NMR (CDCl₃, 300 MHz, ppm): δ 7.44 (s, 2H), 7.28 (d, J=5.1 Hz, 2H), .7.01 (d, J=5.1 Hz, 2H), 3.16 (t, 4H), 2.90 (t, 4H), 2.90 (t, 4H), 1.88 (m, 4H), 1.72 (m, 4H), 1.26 (br, 72H), 0.89 (t, 6H).

¹³C NMR (CDCl₃, 300 MHz, ppm): δ 141.15, 137.99, 136.72, 136.15, 131.58, 128.84, 125.04, 120.56, 33.82, 32.34, 31.27, 30.44, 30.12, 30.08, 30.05, 29.99, 29.94, 29.87, 29.78, 23.10, 14.52.

Synthesis of Polymer 4:

A solution of the 4,8-didodecyl-2,6-bis-(3-dodecyl-thiophen-2-yl)-benzo[1,2-b;4,5-b′]dithiophene monomer 3 (0.41 grams) in 10 mL of chlorobenzene was prepared. 0.32 grams of FeCl₃ and 10 mL of chlorobenzene were placed in a 50 mL round-bottom flask under an argon atmosphere and stirred. While stirring, the solution was added drop-wise through a dropping funnel to the well-stirred mixture over a period of 1 minute. The resulting mixture was stirred at room temperature (about 23 to about 26° C.) for 4 hours under an argon blanket. 15 mL chlorobenzene was added and the solution was put into 200 mL methanol to remove the FeCl₃. The mixture was ultrasonicated for 2 minutes, then stirred at room temperature for 1 hour. The polymer was filtered out of the mixture.

The polymer was then added to a well stirred aqueous solution of 200 mL methanol and 50 mL ammonia (30%). The mixture was subjected to ultrasonication for 30 minutes and then stirred at room temperature for 18 hours. A dark red precipitate was obtained after filtration, which was purified by Soxhlet extraction with methanol for 4 hours and heptane for 24 hours. Chlorobenzene was then used to extract the polymer for 4 hours and a red solution was obtained. Removal of the solvent resulted in 0.35 grams of poly(4,8-didodecyl-2,6-bis-(3-dodecyl-thiophen-2-yl)-benzo[1,2-b;4,5-b′]dithiophene) 4 as a dark red solid. Yield: 85.4%.

The melting point, as measured by DSC, for Example 1 was 286° C. This higher melting point, compared to Comparative Example 1, for the polymer 4 indicated that it had more ordered molecular packing, a crucial property for charge transport. This is believed to be due to the use of the highly pure monomer 3, so that fewer or no structural defects exist in the resulting polymer 4.

### RESULTS

The purity of the monomer 3 for both Comparative Example 1 and Example 1 were determined using HPLC and LC-MS. The results are shown in Table 1.

TABLE 1
HPLC and LC-MS results of monomer 3.

| | HPLC | LC-MS |
|---|---|---|
| Comparative Example 1 | 97.4% | 93.9% |
| Example 1 | 100% | 99.7% |

The monomer of Example 1 had greater purity. The impurities were not completely removed by recrystallization of the monomer from an organic solvent such as isopropanol, hexane, toluene, etc., or by column chromatography using silica gel.

Alumina was a very efficient packing material for column chromatography to separate and remove the impurities from the monomer. Without being bound by theory, it appeared that when hexane was used as an eluent, the impurities had smaller R_{F} (˜0) than the monomer (R_{F}˜0.3), thus allowing the removal of the impurities simply by passing through a column packed with neutral alumina using hexane as an eluent.

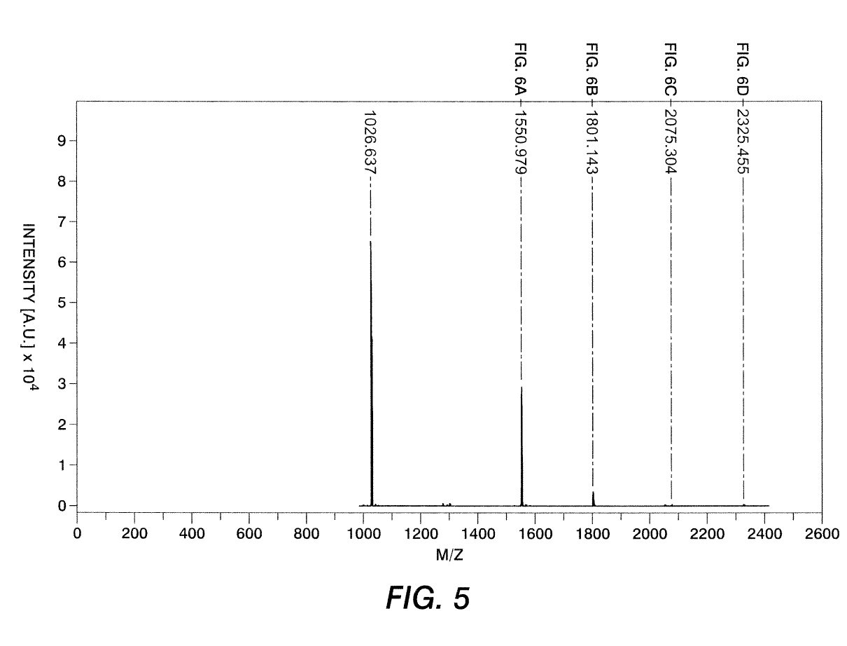

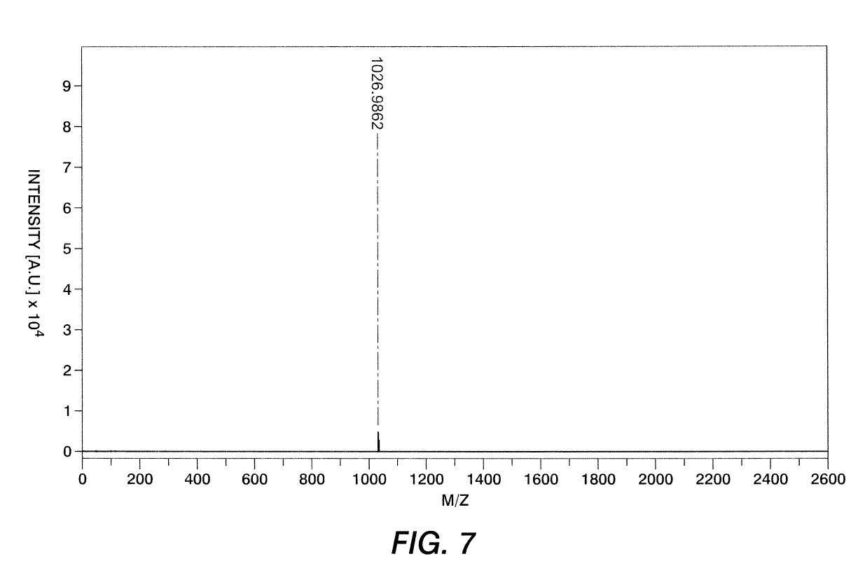

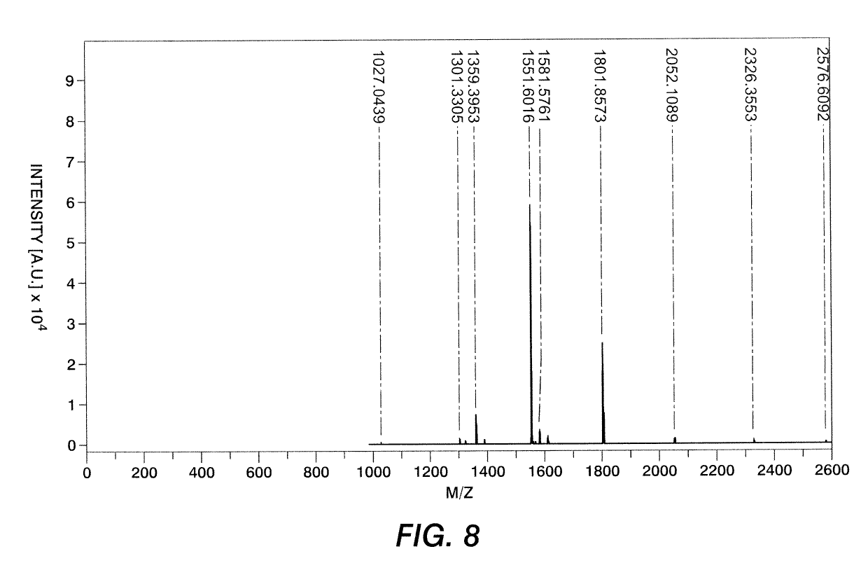

Comparative Example 1 and Example 1 were also examined by MALDI-TOF. FIG. 5 shows the MALDI-TOF spectrum for the product of Comparative Example 1 (collected after being run through the column). FIG. 7 shows the MALDI-TOF spectrum for the product of Example 1 (collected after being run through the column). FIG. 8 shows the MALDI-TOF spectrum for the impurities recovered from the column of Example 1.

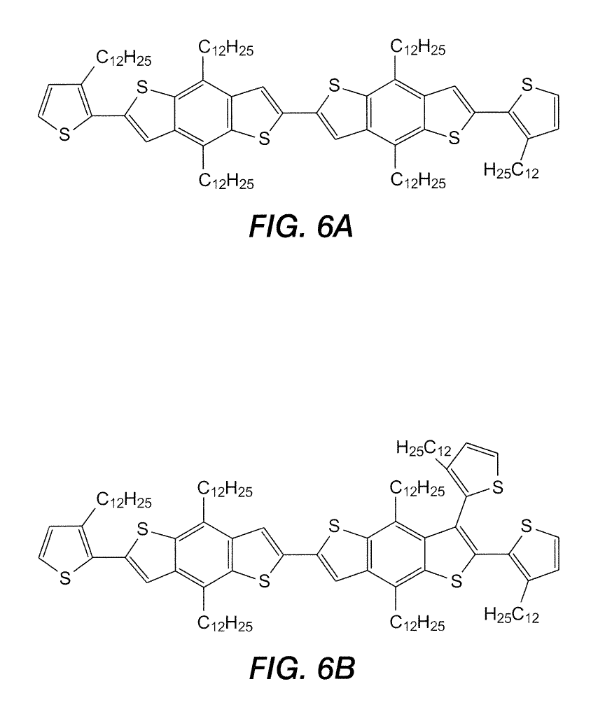

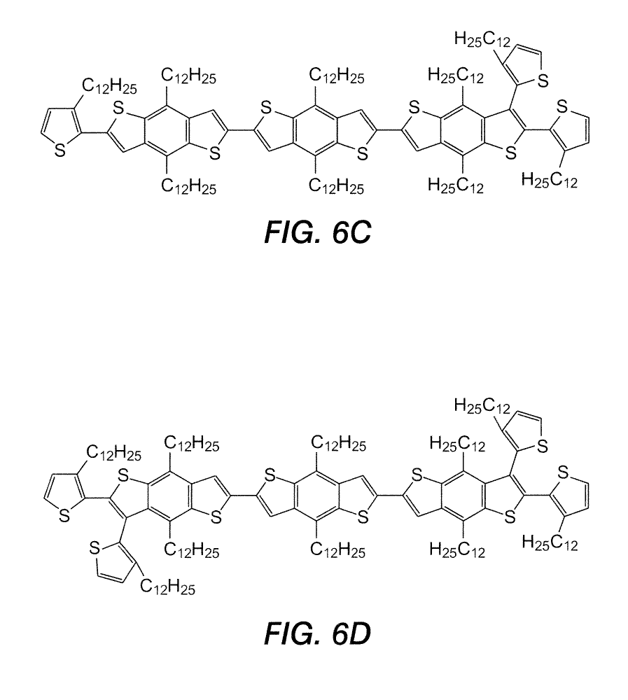

As can be seen in FIG. 5, significant impurities remained in the recovered product. The impurities A-D were separated out, and their structures are also shown in FIG. 6. The main impurities were undesired byproducts shown in FIGS. 6A and 6B.

In comparison, FIG. 7 showed that the monomer of Example 1 had much higher purity; no peaks attributable to impurities are present. Instead, they were trapped in the column, as seen in FIG. 8. Note the peaks in FIG. 8 are in the same location as the peaks for the impurities of FIG. 5.

Next, the polymers of Comparative Example 1 and Example 1 were used to prepare a series of transistors. An n-doped silicon wafer with a 200 nm silicon oxide layer was used as a substrate, the n-doped silicon functioning as the gate electrode and the silicon oxide layer as a gate dielectric layer. The wafer surface was modified with an interfacial layer by immersing the wafer into a solution of 0.1M dodecyltrichlorosilane in toluene at 60° C. for 20 minutes. 10 milligrams of the PBTBT-12 polymer was dissolved in 1 gram dichlorobenzene with heating and filtered through a 0.45 μm syringe filter to form a semiconduct solution. The semiconductor solution was then spin coated onto the wafer substrate at 1000 rpm for 90 seconds. After the solvent was dried off, gold source/drain electrodes were evaporated through a shadow mask on top of the semiconductor layer to complete the OTFT devices. The devices were then characterized with a Keithley 4200-SCS instrument at ambient conditions in the dark. The results are summarized in Table 2.

TABLE 2

| | Mobility (cm²/V · sec) | Current on/off ratio |
|---|---|---|
| Comparative Example 1 | 0.10-0.20 | 10⁶-10⁷ |
| Example 1 | 0.20-0.28 | 10⁶-10⁷ |

The devices made using the polymer of Example 1, with higher purity, had higher mobility. The mobility was also consistently better than the polymer of Comparative Example 1.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A process for purifying a monomer of Formula (II): 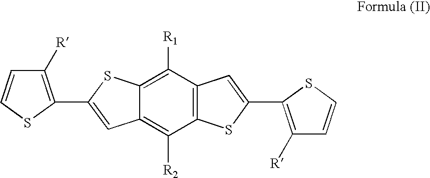
wherein R₁ and R₂ are independently selected from alkyl, substituted alkyl, aryl, substituted aryl, alkoxy, substituted alkoxy, and halogen; and R′ is selected from hydrogen, alkyl, substituted alkyl, aryl, substituted aryl, alkoxy, substituted alkoxy, and halogen; the process comprising:
providing a starting mixture containing the monomer of Formula (II) and impurities; and
passing the starting mixture through a column consisting of neutral alumina using only hexane as an eluent to separate the impurities from the monomer of Formula (II).

2. (canceled)

3. The process of claim 1, further comprising recrystallizing the monomer of Formula (II) from isopropanol, hexane, heptane, or toluene.

4. (canceled)

5. (canceled)

6. The process of claim 1, wherein R₁ and R₂ are identical to each other.

7. The process of claim 1, wherein R₁, R₂, and R′ are identical to each other and are straight chain alkyl having from about 8 to about 18 carbon atoms.

8. The process of claim 1, wherein R₁, R₂, and R′ are C₁₂H₂₅.

9. The process of claim 1, wherein the resulting monomer of Formula (II) has a purity of 98% or greater.

10. The process of claim 1, wherein the resulting monomer of Formula (II) has a purity of 99.5% or greater.

11. A process for preparing a semiconducting polymer with improved mobility, comprising:
providing a starting mixture comprising impurities and a monomer of Formula (II): 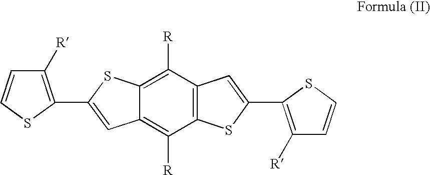
wherein R₁ and R₂ are independently selected from alkyl, substituted alkyl, aryl, substituted aryl, alkoxy, substituted alkoxy, and halogen; and R′ is selected from hydrogen, alkyl, substituted alkyl, aryl, substituted aryl, alkoxy, substituted alkoxy, and halogen;
passing the starting mixture through a column consisting of neutral alumina using only hexane as an eluent to separate the impurities from the monomer of Formula (II); and
polymerizing the monomer of Formula (II) to obtain the semiconducting polymer of Formula (I): 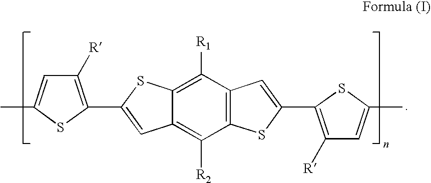
wherein n is the degree of polymerization and is from 2 to about 2,500; and
recrystallizing the monomer of Formula (II) using isopropanol, hexane, heptane, or toluene.

12. (canceled)

13. (canceled)

14. (canceled)

15. The process of claim 11, wherein R₁ and R₂ are identical to each other.

16. The process of claim 11, wherein R₁, R₂, and R′ are identical to each other and are straight chain alkyl having from about 8 to about 18 carbon atoms.

17. The process of claim 11, wherein R₁, R₂, and R′ are C₁₂H₂₅.

18. The process of claim 11, wherein polymerizing the monomer of Formula (II) is carried out using FeCl₃.

19. The process of claim 11, wherein the semiconducting polymer has a mobility of 0.2 cm²/V·sec or greater.

20. The process of claim 11, wherein the semiconducting polymer has a melting point of about 286° C. or greater.

21. (canceled)

22. A process for purifying a BTBT-12 monomer: 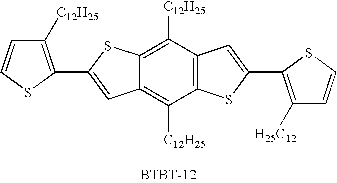
the process comprising:
providing a starting mixture containing the BTBT-12 monomer and impurities;
passing the starting mixture through a column containing neutral alumina using only hexane as an eluent to separate the impurities from the BTBT-12 monomer; and
recrystallizing the BTBT-12 monomer using isopropanol.
